(19) United States
(12) Patent Application Publication
(10) Pub. No.: US 2024/0393204 A1
(43) Pub. Date: Nov. 28, 2024

(54) LEAKAGE DETECTION IN SYRINGES AND SIMILAR SEAL SYSTEMS

(71) Applicant: The Florida State University Research Foundation, Inc., Tallahassee, FL (US)

(72) Inventors: Adam DELONG, Tallahassee, FL (US); Brandon Alexander KRICK, Tallahassee, FL (US); Kylie M. Elizabeth VANMETER, Tallahassee, FL (US); Catherine FIDD, Tallahassee, FL (US); Santiago LAZARTE, Tallahassee, FL (US)

(21) Appl. No.: 18/672,115

(22) Filed: May 23, 2024

Related U.S. Application Data
(60) Provisional application No. 63/504,302, filed on May 25, 2023.

(51) Int. Cl. G01M 3/32; A61M 5/315

(52) U.S. Cl. CPC G01M 3/3281; A61M 5/31513; A61M 2205/15; A61M 2205/3306; A61M 2205/3331; A61M 2209/02

(57) ABSTRACT

A prefilled syringe testing system may include an interface configured to fluidly couple with the cavity of the prefilled sealed container. A prefilled syringe testing system may include a vacuum pump providing a predetermined negative pressure to the interface. A prefilled syringe testing system may include a pressure gauge coupled to the interface and providing a pressure measurement indicative of leakage from the cavity.

### CROSS REFERENCE TO RELATED APPLICATIONS

This application claims the benefit of U.S. Provisional Patent Application No. 63/504,302, filed on May 25, 2023, the entire contents of which are incorporated by reference herein.

### BACKGROUND

This disclosure relates to pre-filled syringes. In particular, this disclosure relates to methods, systems, and apparatuses for determining leakage in pre-filled syringes.

### SUMMARY

In some aspects, the techniques described herein relate to a prefilled container testing system for a prefilled sealed container defining a cavity between a barrel and a stopper, the prefilled container testing system including: an interface configured to fluidly couple with the cavity of the prefilled sealed container; a vacuum pump providing a predetermined negative pressure to the interface; and a pressure gauge coupled to the interface and providing a pressure measurement indicative of leakage from the cavity.

In some aspects, the techniques described herein relate to a prefilled container testing system, wherein the pressure gauge provides an electronic signal indicative of a pressure change.

In some aspects, the techniques described herein relate to a prefilled container testing system, further including a controller configured to receive the pressure measurement from the pressure gauge and determine a leakage condition based on the pressure measurement.

In some aspects, the techniques described herein relate to a prefilled container testing system, wherein a leakage condition is determined when the pressure measurement exceeds a calibratable threshold.

In some aspects, the techniques described herein relate to a prefilled container testing system, further including a housing including a nitrogen atmosphere box.

In some aspects, the techniques described herein relate to a prefilled container testing system, further including a test fixture including the interface, the test fixture defines cooling passages.

In some aspects, the techniques described herein relate to a prefilled container testing system, further including a test fixture including the interface, the test fixture defines heating passages.

In some aspects, the techniques described herein relate to a prefilled container testing system, further including a test fixture including the interface, the test fixture defines a viewing port.

In some aspects, the techniques described herein relate to a prefilled container testing system, further including a camera mounted on a stage configured to visually analyze movement of the stopper via a viewing port.

In some aspects, the techniques described herein relate to a prefilled syringe testing system including: a hermetically sealed housing; a test fixture positioned within the housing and including a syringe interface configured to sealingly receive a prefilled syringe, a viewing port sized to provide a view of a barrel and plunger stopper of the prefilled syringe, a cooling system configured to cool the prefilled syringe, and a heating system configured to heat the prefilled syringe; a vacuum pump fluidly coupled to the syringe interface and providing a predetermined negative pressure; and a pressure gauge fluidly coupled to the syringe interface and providing a pressure measurement indicative of leakage.

In some aspects, the techniques described herein relate to a prefilled syringe testing system, further including a controller configured to receive the pressure measurement from the pressure gauge and determine a leakage condition based on the pressure measurement.

In some aspects, the techniques described herein relate to a prefilled syringe testing system, wherein a leakage condition is determined when the pressure measurement exceeds a calibratable threshold.

In some aspects, the techniques described herein relate to a prefilled syringe testing system, further including a camera mounted on a stage configured to visually analyze movement of the plunger stopper via the viewing port.

In some aspects, the techniques described herein relate to a prefilled syringe testing system, wherein the cooling system is a circulated liquid nitrogen cooling system.

In some aspects, the techniques described herein relate to a prefilled syringe testing system, wherein the hermetically sealed housing includes a nitrogen atmosphere box.

In some aspects, the techniques described herein relate to a method including: filling a syringe with a prescribed fill volume; adjusting a plunger stopper of the syringe to provide a prefilled syringe with a consistent head space; installing the prefilled syringe in a syringe interface; applying a predetermined negative pressure to the prefilled syringe with a vacuum pump; and determining a leakage condition based on a pressure measurement provided by a pressure gauge fluidly coupled to the syringe interface.

In some aspects, the techniques described herein relate to a method, further including: receiving, via a controller, the pressure measurement from the pressure gauge; and comparing, via the controller, the pressure measurement to a predetermined threshold; and determining the leakage condition, via the controller, when the pressure measurement exceeds a calibratable threshold.

In some aspects, the techniques described herein relate to a method, further including: visually analyzing, via a camera, movement of the plunger stopper via a viewing port.

In some aspects, the techniques described herein relate to a method, further including freezing the prefilled syringe via a cooling system.

In some aspects, the techniques described herein relate to a method, further including heating the prefilled syringe via a heating system subsequent to freezing the prefilled syringe.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a front view of a prefilled syringe, according to some implementations.

FIG. 2 is a front view of another prefilled syringe, according to some implementations.

FIG. 3 is a schematic view of a prefilled syringe in an initial state, according to some implementations.

FIG. 4 is a schematic view of the prefilled syringe of FIG. 3 in a freeze state, according to some implementations.

FIG. 5 is a schematic view of the prefilled syringe of FIG. 3 in a first thaw state, according to some implementations.

FIG. 6 is a schematic view of the prefilled syringe of FIG. 3 in a second thaw state, according to some implementations.

FIG. 7 is a schematic view of the prefilled syringe of FIG. 3 in a third thaw state, according to some implementations.

FIG. 8 is a front view of a test prefilled syringe, according to some implementations.

FIG. 9 is a schematic view of a prefilled syringe testing system, according to some implementations.

### DETAILED DESCRIPTION

Following below are more detailed descriptions of concepts related to, and implementations of, methods, apparatuses, and systems for measuring leakage for syringes used in deep cold storage. Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The technology described herein includes systems and methods of measuring leakage, as a function of temperature, for syringes and other similar seal configurations used in deep cold storage. A sealed container of interest includes a seal (e.g., syringe barrel/stopper system) inhibiting ingress of foreign material and egress of sterile solution from the sealed container, and a delivery port (e.g., a luer lock, a needle neck, etc.). A vacuum line is attached to the delivery port and applies a vacuum within the sealed container between the delivery port and the seal. While maintaining a vacuum in the sealed cavity, the sealed container can be cooled and heated with a cryostat. The internal vacuum pressure of the system is recorded with a vacuum gauge (e.g., analog or digital). A change in pressure indicates a change in leak rate of the sealed container via the seal. For example, an increase in pressure indicates leakage. The system can be calibrated by attaching orifices of known geometries (e.g., small tubing or capillary tubes) and modeling the flow rate as a function of pressure drop of each orifice. In a use example, prefilled syringes are used to store and transport biologics like mRNA vaccines at temperatures as low as −80° C. Rubber stoppers in the prefilled syringes form a sterile barrier with the glass or polymer syringe barrel and prevent contamination of the syringe contents. As these syringe systems are cooled, the difference in coefficient of thermal expansion between materials produces separation between the stopper and barrel of the syringe breaking the sterile barrier. This method provides leak detection by measuring the internal pressure.

Syringe leakage can occur with freeze-thaw cycling. The stopper displaces outwardly during freeze-thaw cycling in some systems below a critical temperature. This change in volume and head space is not modeled with a simple ideal gas law. Increases in volume of head space indicates air enters. Leakage onset temperature is a function of system materials, properties and dimensions. Multiple cycling below critical temperature results in multiple stopper displacements. Leakage can be measured by tracking changes in vacuum pressure inside an evacuated syringe. In some implementations, systems and methods disclosed herein give precise temperature range for leakage. In some implementations, systems and methods disclosed herein correlate changes in pressure to optical system changes. In some implementations, systems and methods disclosed herein shows effects of different syringe-stopper-lubricant combinations and the resultant effect on leakage.

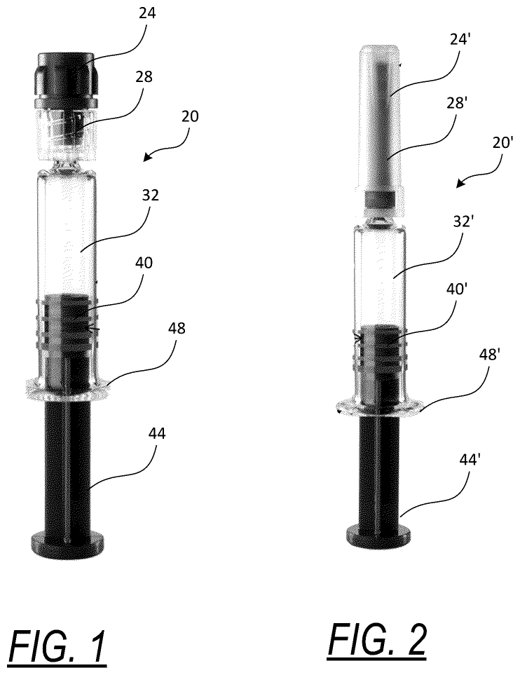

As shown in FIG. 1, a plastic rigid tip cap (PRTC) prefilled syringe 20 includes a PRTC 24 connected to a deliver port 28 in the form of a luer connector. The luer connector is in fluid communication with a barrel 32 that provides a sealing surface for a plunger stopper 40 mounted to a plunger rod 44. The prefilled syringe 20 also includes a flange 48.

As shown in FIG. 2, a rigid needle shield (RNS) prefilled syringe 20′ includes a RNS 24′ connected to a delivery port 28′ in the form of a needle hub. The needle hub is in fluid communication with a barrel 32′ that provides a sealing surface for a plunger stopper 40′ mounted to a plunger rod 44′. The prefilled syringe 20′ also includes a flange 48′.

Other types of prefilled syringes and other types of seal containers are suitable with the systems and methods discussed within this disclosure. The following disclosure will make reference to the prefilled syringe 20 but it is understood that this disclosure contemplates all types of syringes and sealed containers.

Generally, prefilled syringes are filled and sealed by a manufacturer and are ready to use. Syringes can hold biologics, vaccines, anticoagulants, small molecules, etc. The benefits of prefilled syringes include consistent dosing, less injectable waste, improved sterility compared to multi-dose vials, etc. Prefilled syringes have potential uses for storage of sensitive materials like mRNA based vaccines and may be stored at temperatures as low as −80° C. for stability. These storage temperatures can provide challenges such as changes in sealing integrity, thermal expansion, and internal pressure differentials due to freeze thaw cycling. These problems can potentially compromise functionality such as a change in break-loose force, or silicone oil distribution.

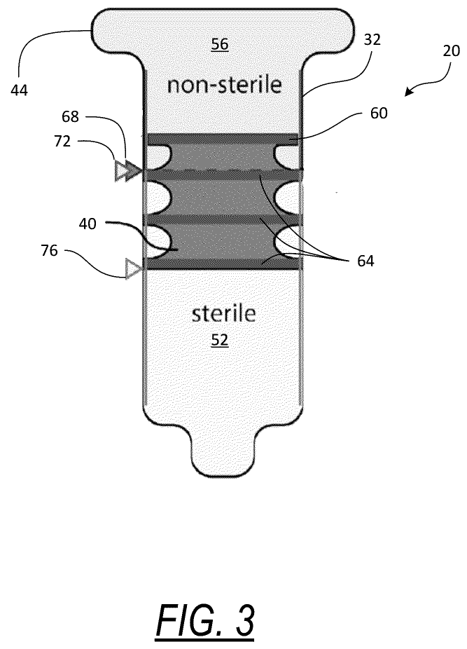

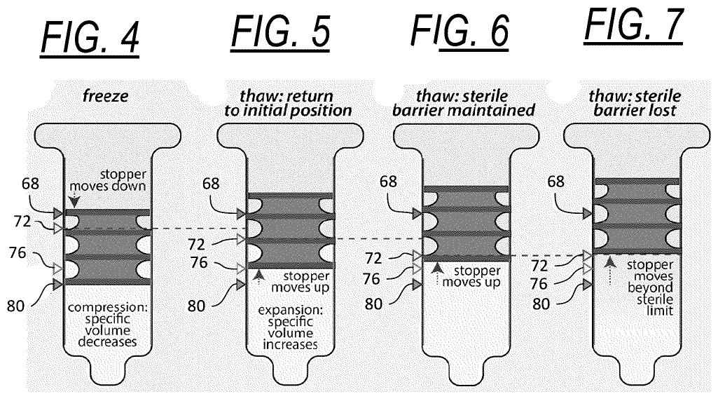

As shown in FIG. 3, in addition to the mechanical functionality and structural integrity of the prefilled syringe 20, container closure integrity is important. CCI protects the drug or contents of the prefilled syringe 20 from foreign matter ingress (leakage), drug product egress including active pharmaceutical ingredients or excipients (leakage), or contact with non-sterile surfaces (Sterile Barrier). The prefilled syringe 20 defines a sterile cavity 52 and a non-sterile cavity 56 that are generally separated by the plunger stopper 40. The plunger stopper 40 includes a trimming ring 60 that does not seal against the interior wall of the barrel 32, and three sealing ribs 64 that inhibit fluid communication between the sterile cavity 52 and the non-sterile cavity 56. The prefilled syringe 20 is shown in FIG. 4 in an initial or reference state with a back initial reference 68 defined at the border between the non-sterile cavity 56 and the sterile cavity 52. In the initial state, a sterile limit 72 is collocated with the back initial reference 68. A front initial reference 76 is defined by a front edge of the plunger stopper 40 in the initial state.

FIGS. 4-7 show movement of the plunger stopper 40 within the barrel 32 during freezing and thawing activities and the resultant effects on the prefilled syringe 20. As shown in FIG. 4, freezing results in movement of the sterile limit 72 away from the back initial reference 68. The compression of the sterile cavity 52 also results in a new maximum inward displacement 80. As shown in FIG. 5, an ensuing thaw event results in the plunger stopper 40 moving outward (e.g., an expansion of the sterile cavity 52) thereby moving the sterile limit 72 to a second of the three sealing ribs 64. This movement results in the expansion of the non-sterile cavity 56 along the plunger stopper 40 toward the sterile cavity 52 while maintaining sterility within the sterile cavity 52. As shown in FIG. 6, further thawing results in additional outward movement of the plunger stopper 40 thereby moving the sterile limit 72 to a third of the three sealing ribs 64. This movement results in the expansion of the non-sterile cavity 56 along the plunger stopper 40 toward the sterile cavity 52 while maintaining sterility within the sterile cavity 52. As shown in FIG. 7, further thawing results in outward movement of the plunger stopper 40 such that all the sealing ribs 64 are past the sterile limit 72 resulting in a loss of sterile environment within the sterile cavity 52. In some implementations, the prefilled syringe 20 includes more than three sealing ribs 64 or less than three sealing ribs 64. The systems and methods discussed herein provide the ability to monitor expansion and contraction of the sterile cavity 52 and thereby to determine when the sterile limit 72 is breached.

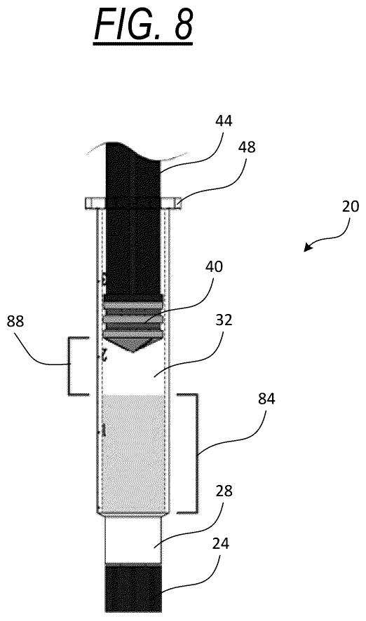

As shown in FIG. 8, the prefilled syringe 20 is prepared for testing conditions and methods by filling the barrel 32 with deionized water to prescribed fill volume 84, and placing the plunger stopper 40 in a controlled position to provide consistent head space 88.

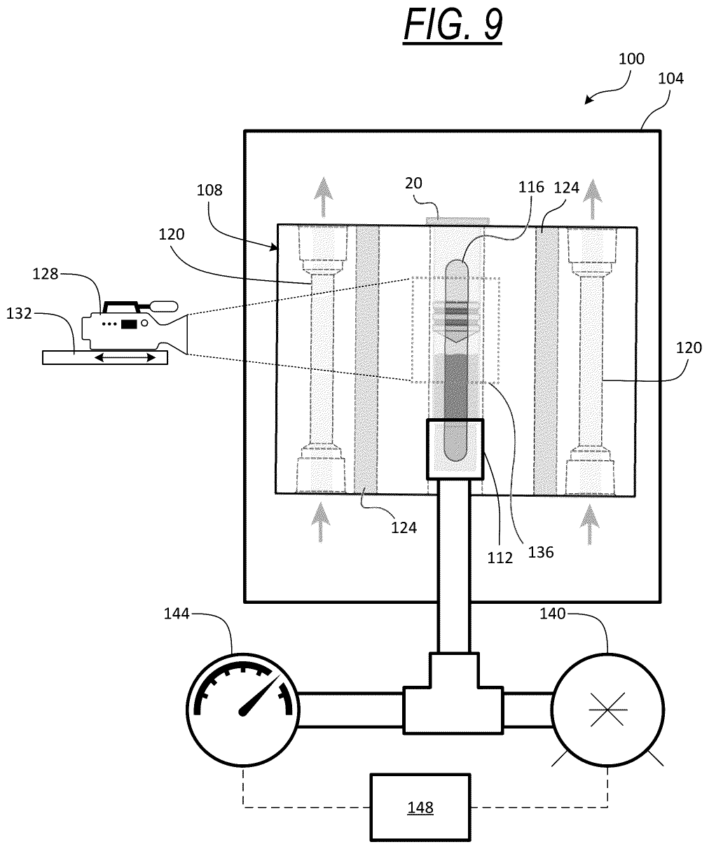

As shown in FIG. 9, a prefilled syringe testing system 100 includes a sealed (e.g., hermetically) housing 104 containing a test fixture 108 structured to hold the prefilled syringe 20. In some implementations, the housing 104 includes a nitrogen atmosphere glove box with ˜0.5 ppm water. The test fixture 108 includes a syringe holder including a syringe interface 112 that sealingly engages the delivery port of the prefilled syringe 20 (e.g., the luer connection) and a viewing port 116 providing a clear view of the sterile cavity 52, non-sterile cavity 56 and position of the plunger stopper 40. In some implementations, the syringe interface 112 includes another type of hermetically sealed connection that connects the prefilled syringe 20 to the prefilled syringe testing system 100. The test fixture 108 includes cooling passages 120 structured to receive a flow of coolant (e.g., liquid nitrogen) to reduce the temperature of the test fixture 108 and thereby the prefilled syringe 20. The test fixture 108 includes heating passages 124 structured to receive a flow of coolant (e.g., glycol) to increase the temperature of the test fixture 108 and thereby the prefilled syringe 20. In some implementations, resistive heaters or another heat source are used. In some implementations, a different cooling system is used. In some implementations, the test fixture 108 is formed from copper.

A camera 128 in the form of a CCD camera (e.g., 4242×2830 pixels, 4.49 μm pixel resolution) is mounted on stage 132 in the form of a stepper motor driven a ball screw stage used to focus the camera 128 on a field of view 136 encompassing the syringe interface 112.

A vacuum pump 140 is fluidly coupled to the syringe interface 112 and provides a predetermined pressure to the syringe interface 112 and a pressure gauge 144 is coupled to the syringe interface 112 and provides a pressure measurement indicative of leakage from the prefilled syringe 20. In some implementations, the pressure gauge 144 provides an electronic signal indicative of a pressure change to a controller 148 that receives the pressure measurement from the pressure gauge 144 and determines a leakage condition based on the pressure measurement. In some implementations, a leakage condition is determined when the pressure measurement exceeds a calibratable threshold. In some implementations, the controller 148 controls operation of a heating system and a cooling system to control a temperature of the test fixture 108.

Data processing conducted by the controller 148 includes pixel locations found for edge of stopper, water, and glass reference point through a duration of the test, and expansion of water during freeze, displacement of stopper, head space changes calculated. Dynamic behavior of prefilled syringe 20 is directly observed and measured during freeze-thaw cycles.

In operation, vacuum pressure can be used to directly measure the leakage in syringes or other sealed containers. Syringes are evacuated to pressure of ˜1 mbar (system and pump at equilibrium pressure, and no aqueous contents in syringe), and the syringe internal pressure is recorded during freeze-thaw cycling. Increases in pressure correspond to system leakage. Leakage can be assumed to occur at a stopper-lubricant syringe barrel interface. External pressure during testing is ˜1 bar.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

What is claimed is:

1. A prefilled container testing system for a prefilled sealed container defining a cavity between a barrel and a stopper, the prefilled container testing system comprising:
an interface configured to fluidly couple with the cavity of the prefilled sealed container;
a vacuum pump providing a predetermined negative pressure to the interface; and
a pressure gauge coupled to the interface and providing a pressure measurement indicative of leakage from the cavity.

2. The prefilled container testing system of claim 1, wherein the pressure gauge provides an electronic signal indicative of a pressure change.

3. The prefilled container testing system of claim 1, further comprising a controller configured to receive the pressure measurement from the pressure gauge and determine a leakage condition based on the pressure measurement.

4. The prefilled container testing system of claim 1, wherein a leakage condition is determined when the pressure measurement exceeds a calibratable threshold.

5. The prefilled container testing system of claim 1, further comprising a housing including a nitrogen atmosphere box.

6. The prefilled container testing system of claim 1, further comprising a test fixture including the interface, the test fixture defines cooling passages.

7. The prefilled container testing system of claim 6, further comprising a test fixture including the interface, the test fixture defines heating passages.

8. The prefilled container testing system of claim 1, further comprising a test fixture including the interface, the test fixture defines a viewing port.

9. The prefilled container testing system of claim 1, further comprising a camera mounted on a stage configured to visually analyze movement of the stopper via a viewing port.

10. A prefilled syringe testing system comprising:
a hermetically sealed housing;
a test fixture positioned within the housing and including
a syringe interface configured to sealingly receive a prefilled syringe,
a viewing port sized to provide a view of a barrel and plunger stopper of the prefilled syringe,
a cooling system configured to cool the prefilled syringe, and
a heating system configured to heat the prefilled syringe;
a vacuum pump fluidly coupled to the syringe interface and providing a predetermined negative pressure; and
a pressure gauge fluidly coupled to the syringe interface and providing a pressure measurement indicative of leakage.

11. The prefilled syringe testing system of claim 10, further comprising a controller configured to receive the pressure measurement from the pressure gauge and determine a leakage condition based on the pressure measurement.

12. The prefilled syringe testing system of claim 10, wherein a leakage condition is determined when the pressure measurement exceeds a calibratable threshold.

13. The prefilled syringe testing system of claim 10, further comprising a camera mounted on a stage configured to visually analyze movement of the plunger stopper via the viewing port.

14. The prefilled syringe testing system of claim 10, wherein the cooling system is a circulated liquid nitrogen cooling system.

15. The prefilled syringe testing system of claim 10, wherein the hermetically sealed housing includes a nitrogen atmosphere box.

16. A method comprising:
filling a syringe with a prescribed fill volume;
adjusting a plunger stopper of the syringe to provide a prefilled syringe with a consistent head space;
installing the prefilled syringe in a syringe interface;
applying a predetermined negative pressure to the prefilled syringe with a vacuum pump; and
determining a leakage condition based on a pressure measurement provided by a pressure gauge fluidly coupled to the syringe interface.

17. The method of claim 16, further comprising:
receiving, via a controller, the pressure measurement from the pressure gauge; and
comparing, via the controller, the pressure measurement to a predetermined threshold; and
determining the leakage condition, via the controller, when the pressure measurement exceeds a calibratable threshold.

18. The method of claim 16, further comprising:
visually analyzing, via a camera, movement of the plunger stopper via a viewing port.

19. The method of claim 16, further comprising freezing the prefilled syringe via a cooling system.

20. The method of claim 19, further comprising heating the prefilled syringe via a heating system subsequent to freezing the prefilled syringe.
